(19) United States
(12) Patent Application Publication
(10) Pub. No.: US 2002/0198574 A1
(43) Pub. Date: Dec. 26, 2002

(54) Automatic sobriety training and reconditioning system

(76) Inventor: Ron Gumpert, Smithtown, NY (US)

Correspondence Address: Jennifer Meredith, Suite 7720, 350 Fifth Ave., New York, NY, 10118, US

(21) Appl. No.: 10/177,359

(22) Filed: Jun. 22, 2002

Related U.S. Application Data
(60) Provisional application No. 60/300,117, filed on Jun. 22, 2001.

(51) Int. Cl. A61N 1/18

(52) U.S. Cl. 607/58

(57) ABSTRACT

The present invention provides a highly efficient method and apparatus for treating substance addiction and for changing addiction-related behavior of users suffering from substance addiction. Specifically, an apparatus is disclosed which senses the presence of a substance and transmits information regarding its presence to a processor and optionally a care giver. The user may also be given electrical stimulus through the apparatus. The electrical stimulus may be increased according to the amount of a substance present.

### RELATED CASES

This application is related to and claims the filing date of provisional patent application No. 60/300,117 filed Jun. 22, 2001.

### BACKGROUND OF THE INVENTION

The present invention generally relates to a method and apparatus for the diagnosis, prevention and treatment of substance abuse.

Nicotine, cocaine, and other addictive substances are readily available and routinely used by large segments of the United States population. Addiction can occur by use of legal and illegal substances. Substance abuse and addiction is a powerful and debilitating affliction affecting not only the abuser, but the family and friends of the abuser. Often the abuser is out of control and lacks the ability to overcome his addiction.

As used herein, addiction-related behavior means behavior resulting from compulsive substance use and is characterized by apparent total dependency on the substance. Typically a substance abuser experiences a pleasurable feeling in the beginning of the usage. After the effects have worn off, a user often craves more of the substance. These cravings may be intense and are often so severe that they cause gastrointestinal rumblings, spasms, seizure-like convulsions, and many other symptoms. By way of example, a cocaine user experiences three stages of drug effects. The first, acute intoxication (“binge”), is euphoric, marked by decreased anxiety, enhanced self-confidence and sexual appetite, and often marred by sexual indiscretions, irresponsible spending, and accidents attributable to reckless behavior. The second stage, the “crash”, replaces euphoria with anxiety, fatigue, irritability and depression. Many users have committed suicide during this period. Finally, the third stage, “anhedonia,” is characterized by a limited ability to derive pleasure from normal activities and by craving for the euphoric effects of cocaine. See Gawin and Kleber, Medical Management of Cocaine Withdrawal, 6-8 (APT Foundation). Most addiction-related behavior includes behavior associated with all three stages of drug effects. Symptomatic of addiction is (i) overwhelming involvement with the use of the drug, (ii) the securing of its supply, and (iii) a high probability of relapse after withdrawal.

Pharmacological treatment, counseling, psychological assistance, and psychiatric treatment are just some of the methods used to treat addiction. However, the rate of success for permanently breaking such addictions is relatively low for any heretofore known method or type of treatment. Many addicts relapse after long and expensive periods of anti-addiction treatment and therapy. Thus, while there are many treatments for addiction known within the art, none of them have been shown to be truly effective. Perhaps, this is because of their inability to provide 24 hours a day, seven day a week monitoring, combined with feedback and reporting of the presence of substances.

U.S. Pat. No. 5,086,058 discloses a method for treating alcoholism. Specifically, the alcohol-drinking response of alcoholics is extinguished by having them drink alcoholic beverages while nalmefene, an opiate antagonist, blocks the positive reinforcement effect of ethanol in the brain. This method includes continued medication until withdrawal symptoms occur and the patient is then treated in the normal manner for withdrawal. The problem with such methods is two fold. First, there is not constant supervision. Second, there is nothing to trigger treatment of the underlying problems when a recurrence may occur.

Substance monitoring systems are also currently needed to measure a subject's blood alcohol level at a remote location over an extended period of time, such as when a subject is in an alcohol rehabilitation program, or is under court to refrain from consuming alcohol or drugs. For this type of system, identification of the subject providing the sample for testing is a significant problem. For example, an unsupervised remote breath alcohol tester being used by the subject is ineffective if an operator is not available to identify that the correct subject is breathing into the instrument. Because of the requirement of subject identification, these systems typically require the active participation and cooperation of the subject. Sometimes the subjects are uncooperative and will go to great lengths to avoid the testing. This increases the time and effort necessary to perform the testing, which often results in less testing being performed.

Another problem is that once a subject has been tested, the subject knows they will not be tested again for some period of time, such as when the subject is involved in a work release or outpatient rehabilitation program. This allows the subject to leave the location of the stationary remote tester unit and immediately consume substances at high levels just after the testing. The subject is unlikely to show a high level of alcohol or drug content if tested at a much later time because the rate of dissipation of alcohol and certain drugs from the human body is quite high.

It would be desirable to not only detect and report the presence of a substance constantly, but to report its presence to a caregiver, and provide therapy. Transcutaneous Electrical Stimulation is known within the art as a therapeutic tool. By way of example, Transcutaneous Electrical Feedback has been used to treat sleep problems, attenuation of withdrawal from drug addiction, relief from asthma, and relief from anxiety and depression. U.S. Pat. No. 4,503,863, herein incorporated by reference, discloses a Method and Apparatus for Transcutaneous Electrical Stimulation. The '863 patent discloses obtaining actual bio-electrical characteristics of a subject under predetermined conditions of evoked response stimuli, and by interaction with a computer, applying cutaneous electrical stimulation to the subject, using a signal generator to modify current amplitude and frequency in a direction to achieve bio-electrical characteristics in the subject related to the actual bio-electrical characteristics monitored. The signal generator uses a sinusoidal waveform output, with battery power passing through a transformer to power a transconductance amplifier to obtain constant current output despite resistance changes in the line with the subject, and with the transformer not placed in the signal path of the sinusoidal waveform. The signal may be obtained from a sinusoidal oscillator of wide frequency having switched integrator sections connected to an inverting amplifier, pulse generator and gating. This method and apparatus is generally voluntary and an in-house treatment, which is to say is accomplished in a hospital or under the care of a physician. It would be desirable to have a system in which the use of a substance may be detected, treatment, such as an electrical stimulus or medication is delivered to the user, and finally a physician, law enforcement, parent or other individual of the substance abuse by the user. Such a system would allow for constant supervision, which could allow for substance abusers to be released from jail and treatment centers, yet still monitored. At the same time, the abuser is receiving treatment.

As can be seen, there is a need for a method and apparatus that monitors a user 24 hours a day, seven days a week, provides feedback and reporting of the presence of substances, and at the same time provides treatment to the user. Also, the user may be monitored and delivered medication according to the levels of addiction and cravings. While monitoring, medication and counseling are known methodologies, they have not been effectively combined to offer a method and apparatus that provides constant supervision, medication and monitoring such as is needed for the treatment of substance abuse, as disclosed herein.

### SUMMARY OF THE INVENTION

The present invention is directed to a method and apparatus for treating addictions, maladies and the like. Specifically, an apparatus is provided that allows for the constant monitoring for the presence of a substance, feedback to the user, treatment and reporting to another party.

One aspect of the invention is an apparatus for monitoring and sensing substance abuse by a user and providing feedback and treatment to said user, comprising of power supply for providing electrical power, a substance sensor for measuring a percentage of a given substance within the user's bloodstream, an attachment device for attaching said apparatus to said user, a transmitter for transmitting information regarding said percentage of a given substance within said user's bloodstream, a processor for receiving information regarding said percentage of a substance within said user's bloodstream, and an electrical stimulus device for administering an electrical stimulus when said percentage of a given substance exceeds a given level.

According to another aspect of the present invention, a method of sensing the presence of a substance, providing feedback and reporting the presence of a substance to a caregiver is disclosed comprising the steps of attaching a removable apparatus to a user, monitoring the presence of at least one substance within the blood stream of the user to determine if the presence of the given substance exceeds a percentage, and transmitting data confirming the presence of the substance to a processor, wherein the processor activates an electrical stimulus device to deliver an electrical stimulus to the user. Also, the processor reports to the caregivers of the users presence of the substance.

According to a further aspect of the present invention, a method of treating substance abuse is disclosed comprising the steps of attaching an apparatus to a user so that a caregiver may remove the apparatus and the user may not. The apparatus may have an attachment device, a lock, and a battery in electrical communication with a substance sensor, an electrical stimulus device and a processor. The level of a substance within the blood stream may be determined if the presence of the substance within the user's bloodstream is greater than a given level. An electrical stimulus may be delivered to the user when the presence of the substance within a user's bloodstream is greater than a given level. The apparatus may communicate to the caregiver the presence of the substance within the user's bloodstream. The level of the substance within the user's blood stream may be reassessed and an increased electrical stimulus delivered up to a maximum amount where the level of the substance within the user's blood stream has increased. The levels and amounts of electrical stimulus may be reported to a caregiver. The caregiver may determine whether to medicate the user, and the processor may also make a determination and suggestions.

According to another aspect of the present invention, a method of treating substance abuse is disclosed comprising the steps of removably attaching an apparatus to a user so that a caregiver may remove the apparatus, but the user may not. The apparatus may comprise an attachment device, a lock, and a battery in electrical communication with a substance sensor, an electrical stimulus device and a processor. The method comprises the steps of providing a unique individual profile or medical record for the user, which provides maximum electrical stimulations levels, given acceptable levels of a substance within a user's bloodstream, drug interactions, drug prescriptions, and preferred medications. The level of a substance within the blood stream may be determined to see if the presence of a substance within the user's bloodstream is greater than a given level, A preferred level of electrical stimulus may be delivered to the user when the presence of the substance within a users bloodstream is greater than a given level. The presence of the substance within the users bloodstream may be communicated to the caregiver. Then level of the substance within the user's blood stream may be assessed again. If the level of the substance within the user's blood stream has increased, an increased electrical stimulus may be delivered, up to a maximum amount. The level of the substance and the level of electrical stimulus delivered to the user may be reported to a caregiver. An assessment may be made as to whether medication is desired and the appropriate medication may be determined. The medication may be automatically and securely delivered to the user.

These and other features, aspects and advantages of the present invention will become better understood with reference to the following drawings, description and claims.

### BRIEF DESCRIPTION OF THE DRAWINGS

FIG. 1 is a side view of the present invention according to a preferred embodiment;

FIG. 2 is a side view of the present invention according to a preferred embodiment;

FIG. 3 is a side view of the present invention according to a preferred embodiment;

FIG. 4 is a schematic of the present invention according to a preferred embodiment; and

FIG. 5 is a flowchart according to a method of the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention.

The present invention provides a highly efficient method and apparatus for treating substance addiction and for changing addiction-related behavior of users suffering from substance addiction. As used herein, addiction-related behavior means behavior resulting from compulsive substance use and is characterized by apparent total dependency on the substance. Symptomatic of the behavior is (i) overwhelming involvement with the use of the drug, (ii) the securing of its supply, and (iii) a high probability of relapse after withdrawal.

By way of example, a cocaine user may experience three stages of drug effects. The first, acute intoxication (“binge”), is euphoric, marked by decreased anxiety, enhanced self-confidence and sexual appetite, and often marred by sexual indiscretions, irresponsible spending, and accidents attributable to reckless behavior. The second stage, the “crash”, replaces euphoria with anxiety, fatigue, irritability and depression. Many users have committed suicide during this period. Finally, the third stage, “anhedonia,” is characterized by a time of limited ability to derive pleasure from normal activities and by craving for the euphoric effects of cocaine. See Gawin and Kleber, Medical Management of Cocaine Withdrawal, 6-8 (APT Foundation). Most addiction-related behavior includes all three stages of drug effects.

Drugs of abuse include but are not limited to those acting as control stimulants such as alcohol, cocaine, amphetamine, pipradol, methylphenidate, nicotine and caffeine. Drugs of abuse also include narcotics such as morphine and methadone, as well as CNS depressants such as barbiturates, chlordiazepoxide and ethanol. Compulsive drug use includes three independent components: tolerance, psychological dependence and physical dependence. Tolerance produces a need to increase the dose of the drug after several administrations in order to achieve the same magnitude of effect. Physical dependence is an adaptive state produced by repeated drug administration and which manifests itself by intense physical disturbance when drug administration is halted. Psychological dependence is a condition characterized by an intense drive or craving for a drug the effects of which the user feels are necessary for a sense of well being. See Feldman, R. S. and Quenzer, L. F. “Fundamentals of Neuropsychopharmocology” 418-422 (Sinaur Associates, Inc.) 1984 incorporated herein by reference as if set forth in full. Based on the foregoing definitions, as used herein “dependency characteristics” include all characteristics associated with compulsive drug use, characteristics that can be affected by biochemical composition of the host, and physical and psychological properties of the host.

As explained above, the compulsive use of drugs of abuse and gives rise to a euphoric stage followed by a stage of craving for the euphoric effects of that drug. As used herein the rewarding/incentive effects of drugs of abuse refers to any stimulus (in this case, a drug) that produces hedonia or increases the probability of a learned response. This is synonymous with reinforcement. A stimulus is deemed to be rewarding by using paradigms that are believed to measure reward.

The present invention provides a method of treating substance abuse and is disclosed comprising the steps of removably attaching an apparatus to a user so that a caregiver may remove the apparatus, but the user may not. The apparatus may comprise an attachment device, a lock, and a battery in electrical communication with a substance sensor, an electrical stimulus device and a processor.

The attachment device may be a wristband, earring, nose ring, tongue ring, ankle bracelet, arm bracelet or any other method known within the art.

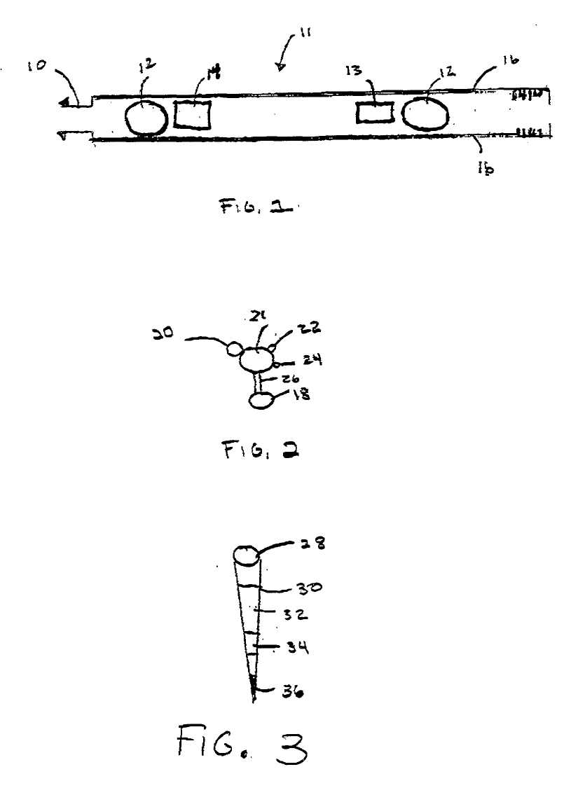

FIG. 1 depicts an apparatus according to the present invention. As shown, the wristband 11 may have a lock 10, a substance sensor 12, a transmitter 13, and a power means 14 which may be a lithium rechargeable battery. The electrical stimulus may be delivered through an electrode tape 16 along the edge of the apparatus. There may be a number of different apparatus without departing from the scope of the invention. There may also be an amplifier or transducer.

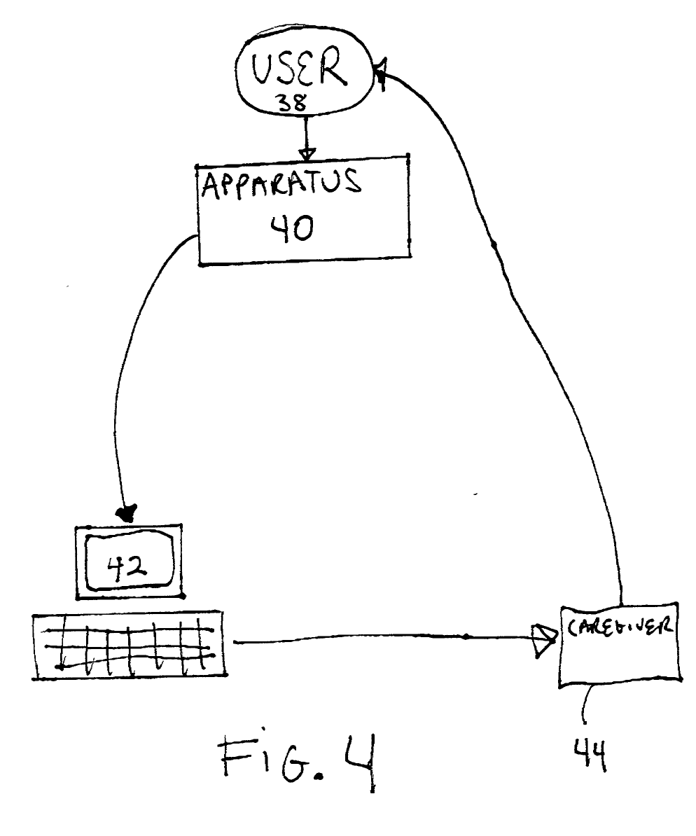

FIG. 2 depicts the apparatus as a tongue ring. As shown, there may be a top portion 21, with an amplifier 20 attached to the top portion 21, an electrode 22, and alcohol sensor 24. The apparatus may have a pin 26 that goes through the tongue and has electrodes to create an electrical stimulus along the side 26. The power means 18 may also act as a lock. It should also be noted that if any of the devices are ever tampered with the transmitter may transmit a message to the processor thereby sending a message to a caregiver. The caregiver may be law enforcement, a parent or any person who has an interest in the user.

The attachment device may also be an implant. In severe cases, where the substance abuser is sabotaging efforts to cure their addiction, it may be necessary to implant the device. By way of example, it may be desirable to implant the device in between the shoulder blades of the user. In that way, they cannot easily tamper with the device. FIG. 3 depicts an implant. As shown, there may be an alcohol sensor 28, that may be part of a top portion that may be removably attached to a bottom portion. The top portion may be screwed onto the bottom portion as shown, where 30 depicts the threading between the top portion and the bottom portion. Within the bottom portion there may be a compartment for containing a battery 32. The bottom portion may also contain a transmitter, transducer, or amplifier. There may also be an electrode 36 at the bottom of the apparatus that provides electrical stimulation when the presence of a substance above a certain level is detected in the users blood stream.

The lock may be a simple key lock, or may be digital, or of any other means wherein a person may lock a device. It may also be a securing means, that transmits a message to the caregiver in the event that the user attempts its removal. As shown in FIG. 4, the user 38 has the apparatus 40 attached to him. The apparatus monitors the user, and delivers information to a processor which may be a computer 42. The computer 42 may then, in turn, deliver information to the caregiver 44. The caregiver 44 may then check on the user 38.

Substance sensors take many forms and may include a conducting/nonconducting regions sensor, a SAW sensor, a quartz microbalance sensor, a conductive composite sensor, a chemiresistor, a metal oxide gas sensor, an organic gas sensor, a MOSFET, a piezoelectric device, an infrared sensor, a sintered metal oxide sensor, a Pd-gate MOSFET, a metal FET structure, an electrochemical cell, a conducting polymer sensor, a catalytic gas sensor, an organic semiconducting gas sensor, a solid electrolyte gas sensors, a piezoelectric quartz crystal sensor, a transdermal patch sensor, fuel cells, electrocardiogram, a a blood pressure sensor, a video camera, a breath sensor and any combinations thereof.

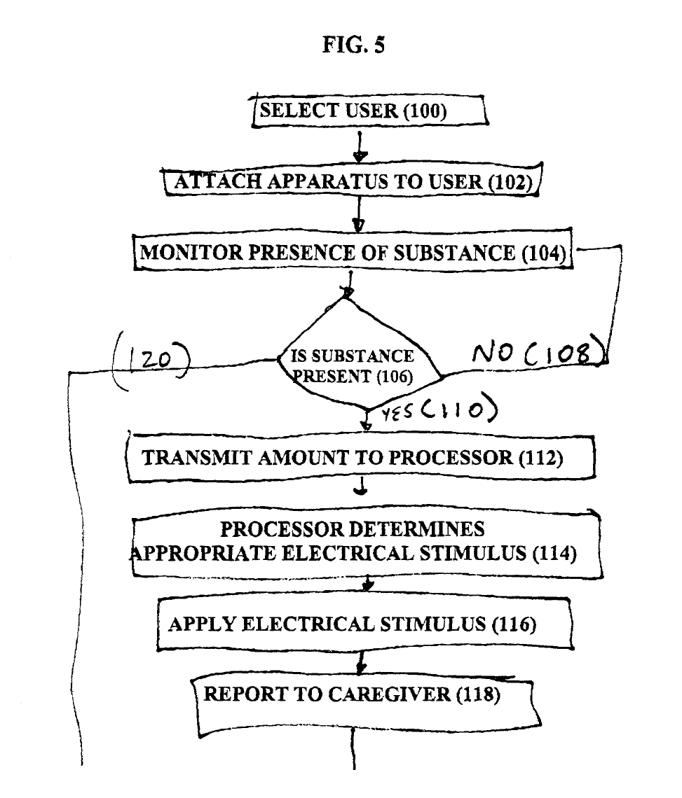

FIG. 5 depicts a method of treating substance abuse according to the present invention. As shown, a user may be selected (100) and an apparatus attached to the user (102). The apparatus constantly monitors the presence of a substance (104) in the user's bloodstream. Constantly, it asks is the substance present (106)? If no (108) the monitoring continues. If yes (110), the amount present is transmitted to the processor (112). The processor determines the appropriate electrical stimulus (114).

A unique individual profile or medical record may be created for the user, which provides maximum electrical stimulations levels, given acceptable levels of a substance within a users bloodstream, drug interactions, drug prescriptions, and preferred medications. The level of a substance within the blood stream may be determined to see if the presence of a substance within the users bloodstream is greater than a given level. By way of example, the substance sensor may detect the presence of alcohol vapor present on the skin. This is to point out that while the substance sensor is intended to dense the amount of a substance present in the blood stream of the user, it may monitor or sense the presence of a substance in a number of different ways.

When it is determined that a user has a blood alcohol content greater than a certain level or percentage, this may trigger an electrical stimulus. A preferred level of electrical stimulus may be delivered to the user when the presence of the substance within a users bloodstream is greater than a given level. For example, the computer can be programmed to automatically be able to compare and contrast different combinations and permutations of different frequencies of electrical stimulation to determine which has the most efficient output in evoking the recruitment of synchronization of EEG activity. The latter is associated with a pleasurable or sedated state of consciousness.

Any methods known within the art to deliver electrical stimulation may be used. This may be electrodes or any other electrical stimulus (116). The stimulus is intended to serve as a “wake up call” to the abuser. Also, the stimulus provides negative associations with the addiction. In this way, the electrical stimulus provides negative associations with the drug, gives them a wake-up call, and informs their caregiver (who may be a police officer, doctor, parent, or the like). Electrical Stimulation as a high frequency continuous mild electrical activity may also block the pain signal traveling to the brain. If the pain signal does not get through to the brain, the pain is not “felt”. Also, electrical stimulation works by stimulating the body's own natural pain-control mechanism. Low frequency or short bursts of electrical activity causes the body to release its own pain relieving substances, called endorphins. The frequency may be between 3 and 800 hertz.

The presence of the substance within the users bloodstream may also be communicated to the caregiver (118). In this way, outside help may be brought in. Also, the user, fearing the repercussions of the caregiver being made aware of their use, will avoid using and abusing substances. The level of the substance within the users blood stream may be assessed again (120) and again this information is transmitted to the processor (112). If the level of the substance within the users blood stream has increased, an increased electrical stimulus may be delivered, up to a maximum amount. The level of the substance and the level of electrical stimulus delivered to the user may be reported to a caregiver. It may be assessed whether medication is desired and the appropriate medication determined. The medication may be automatically and securely delivered to the user. This may be accomplished through the use of a special dispenser in communication with the present invention and to be disclosed in further detail in a future patent.

It should be understood, of course, that the foregoing relates to preferred embodiments of the invention and that modifications may be made without departing from the spirit and scope of the invention as set forth in the following claims.

We claim:

1. An apparatus for monitoring and sensing substance abuse by a user and providing feedback and treatment to said user comprising;
a power supply for supplying power;
a substance sensor for measuring a percentage of a given substance within said user's bloodstream;
a locking attachment device for attaching said apparatus to said user, wherein said locking attachment device is secured by a caregiver such that said caregiver may remove said apparatus and user may not remove said apparatus;
a transmitter for transmitting information regarding said percentage of a given substance within said user's bloodstream;
a processor for receiving information regarding said percentage of a substance within said user's bloodstream; and
an electrical stimulus device for administering an electrical stimulus when said percentage of a given substance exceeds a given level.

2. An apparatus as in claim 1, wherein said substance sensor is chosen from the group consisting of conducting/nonconducting regions sensor, a SAW sensor, a quartz microbalance sensor, a conductive composite sensor, a chemiresistor, a metal oxide gas sensor, an organic gas sensor, a MOSFET, a piezoelectric device, an infrared sensor, a sintered metal oxide sensor, a Pd-gate MOSFET, a metal FET structure, a electrochemical cell, a conducting polymer sensor, a catalytic gas sensor, an organic semiconducting gas sensor, a solid electrolyte gas sensors, and a piezoelectric quartz crystal sensor, a transdermal patch sensor, fuel cells, an electrocardiogram, a blood pressure sensor, a video camera, a breath sensor and any combinations thereof.

3. An apparatus as in claim 1, wherein said attachment device is chosen from the group consisting of an implant, wristband, earring, nosering, tongue ring, ankle bracelet, arm bracelet, wrist bracelet and any combination thereof.

4. An apparatus as in claim 1, wherein said electrical stimulus has a frequency between 3 and 800 hertz.

5. An apparatus as in claim 1, further comprising an amplifier or transducer.

6. A method of sensing the presence of a substance, providing feedback and reporting the presence of a substance to a caregiver comprising the steps of:
removably attaching an apparatus to a user, wherein said apparatus comprises an attachment device, a substance sensor, a transmitter, a processor and an electrical stimulus device;
monitoring the presence of at least one substance within the blood stream of said user through said substance sensor to determine if the presence of a given substance within a users bloodstream exceeds a chosen percentage;
if the percentage of a given substance exceeds a chosen percentage, transmitting data confirming the presence of a percentage of a given substance through a transmitter to a processor, wherein said processor activates said electrical stimulus device to deliver an electrical stimulus to said user; and
reporting the presence of said substance within said users bloodstream to said caregiver.

7. A method as in claim 6, further comprising increasing the amount of electrical stimulus delivered to said user, up to a maximum level, when the amount of a given substance within said users bloodstream increases.

8. A method as in claim 6, wherein said electrical stimulus is transcutaneous electrical stimulation with a frequency between 3 and 800 hertz.

9. A method as in claim 6, further characterized by providing a unique individual profile or medical record of the frequency versus threshold current intensity at which the user feels the subjective sensation of electrical stimulation being applied.

10. A method as in claim 6, further characterized by said processor being a computer, which may interactively monitor the presence of a substance within said user, administer electrical stimulation according to the amount of a substance present, prescribe medication and automatically and securely deliver said medication.

11. A method as in claim 6, wherein said attachment device further comprises a locking means wherein said caregiver removably attaches said apparatus to said user such that said caregiver may remove said apparatus and said user may not remove said apparatus.

12. A method as in claim 6, further comprising automatically delivering medication to said user.

13. A method as in claim 6, wherein said medication is chosen from the group consisting of disulfiram, antabuse, chiorpromazine, thorazine, oxycontin, percocet, darvon, darvocet, vicodin, lorcet, dialaudid, demerol, nalmefene, valium, librium, Xanax, halcyon, Pro Som, and medicament including gamma vinyl GABA.

14. A method of treating substance abuse comprising the steps of;
removably attaching an apparatus to a user so that a caregiver may remove said apparatus and user may not remove said apparatus, said apparatus comprising an attachment device, a lock, and a battery in electrical communication with a substance sensor, an electrical stimulus device and a processor;
determining the level of a substance within the blood stream to determine if the presence of said substance within said users bloodstream is greater than a given level;
delivering a preferred level of electrical stimulus to said user when the presence of said substance within a users bloodstream is greater than a given level;
communicating to said caregiver the presence of said substance within said users bloodstream;
determining the level of said substance within said users blood stream;
delivering an increased electrical stimulus, up to a maximum amount, where the level of said substance within said users blood stream has increased;
reporting the level of said substance and the level of electrical stimulus delivered to said user to a caregiver; and
assessing whether medication is desired.

15. A method as in claim 14, further comprising determining appropriate medication and automatically and securely delivering said medication to said user.

16. A method as in claim 14, wherein said electrical stimulus has a frequency between 3 and 800 hertz.

17. A method as in claim 14, further characterized by providing a unique individual profile or medical record for said user which provides maximum electrical stimulations levels, given acceptable levels of a substance within a users bloodstream, drug interactions, drug prescriptions, preferred medications and the frequency versus threshold current intensity at which the user feels the subjective sensation of electrical stimulation being applied.

18. A method as in claim 14, further characterized by said processor being a computer, which may interactively monitor the presence of a substance within said user, administer electrical stimulation according to the amount of a substance present, prescribe medication and automatically and securely deliver said medication.

19. A method as in claim 14, further comprising automatically delivering medication to said user.

20. A method as in claim 14, wherein said medication is chosen from the group consisting of disulfiram, antabuse, chlorpromazine, thorazine, oxycontin, percocet, darvon, darvocet, vicodin, lorcet, dialaudid, demerol, nalmefene, valium, librium, Xanax, halcyon, Pro Som, and medicament including gamma vinyl GABA.

21. A method of treating substance abuse comprising the steps of;
removably attaching an apparatus to a user so that a caregiver may remove said apparatus and user may not remove said apparatus, said apparatus comprising an attachment device, a lock, and a battery in electrical communication with a substance sensor, an electrical stimulus device and a processor;
providing a unique individual profile or medical record for said user, which provides maximum electrical stimulations levels, given acceptable levels of a substance within a users bloodstream, drug interactions, drug prescriptions, and preferred medications;
determining the level of a substance within the blood stream to determine if the presence of said substance within said users bloodstream is greater than a given level;
delivering a preferred level of electrical stimulus to said user when the presence of said substance within a users bloodstream is greater than a given level;
communicating to said caregiver the presence of said substance within said users bloodstream;
determining the level of said substance within said users blood stream;
delivering an increased electrical stimulus, up to a maximum amount, where the level of said substance within said users blood stream has increased;
reporting the level of said substance and the level of electrical stimulus delivered to said user to a caregiver;
assessing whether medication is desired;
determining appropriate medication; and
automatically and securely delivering said medication to said user.
